(19) United States
(12) Patent Application Publication
(10) Pub. No.: US 2008/0195023 A1
(43) Pub. Date: Aug. 14, 2008

(54) BLOOD OPTIMIZATION PROGRAM

(75) Inventor: Joseph Feldschuh, Bronx, NY (US)

Correspondence Address: LACKENBACH SIEGEL, LLP, LACKENBACH SIEGEL BUILDING, 1 CHASE ROAD, SCARSDALE, NY, 10583, US

(73) Assignee: Daxor Corporation, New York, NY (US)

(21) Appl. No.: 11/673,062

(22) Filed: Feb. 9, 2007

(51) Int. Cl. A61M 1/38; A61M 1/36

(52) U.S. Cl. 604/6.03; 604/4.01

(57) ABSTRACT

Method of optimization of blood and system for customizing blood for future use of an individual which includes: obtaining a unique blood volume measurement from the individual or a potential blood donor; administering an epoetin alfa medication to the individual or potential blood donor for stimulating bone marrow to increase production of red blood cells in accordance with information obtained from the measurement of the blood volume of the individual or the potential blood donor; and storage of blood obtained from the individual or potential blood donor if the blood donor is qualified in accordance with acceptable medical procedures to donate blood.

### BACKGROUND OF THE INVENTION

This invention is concerned with optimization of an individual's blood volume and a Blood Optimization Program to provide blood for future use. More particularly, the invention is concerned with the use of blood during surgery and for storage purposes for later usage.

### DESCRIPTION OF THE PRIOR ART

As is well known, there are significant risks from donor blood transfusions. These risks include infections from diseases such as hepatitis or AIDS, which clearly are not completely detectable by the current screening methods. For example, a screening period for AIDS may be as long as six (6) months to detect for HIV and up to one (1) year for various strains of viral hepatitis. While these diseases have been reduced, a donor may still be infected during what is called a “window of non-detectability” wherein infected donors will test negative.

Other diseases, including certain types of hepatitis, are not tested by current screening processes. Diseases such as cytomegalovirus (CMV), which affects an estimated 10-20% of the population, are not currently screened. Another potentially fatal disease, seen primarily in South American and Hispanic donors known as Chagas Disease, is not currently screened for. Chagas Disease causes irreversible heart failure. Mad cow disease, also known as Bovine Spongiform Encephalitis (BSE) is not screened for at the present time, and current plans by regulators are to exclude all Americans as donors who have spent any significant period of time in Europe over the past 20 years. Mad cow disease, however, is believed to be present in elk in the west, and there is evidence that this disease is, therefore, present in Americans who also are potential blood donors. BSE is known to cause dementia and may have symptoms which may be almost identical to Alzheimer's disease.

Donor blood also causes a reaction to the blood known as immunomodulation, often resulting in depression of the recipient's immune system. Roughly, exposure to components of the donor's immune system in the donor's blood causes the recipient's immune system to be altered in response. This has been associated with an increased risk of infection following transfusion and, in patients being operated on for cancer, an increased risk of recurrence of the cancer. Filtering out white blood cells from donated blood reduces immtmomodulation and thus reduces the risks for these adverse events.

Lack of blood transfusions when someone is severely blood depleted may result in strokes, heart attacks, memory loss, and even death. In recent years, medical practice has changed so that blood transfusions are administered only to patients who show severe degrees of blood loss. A standard test for decision making in regard to administering a blood transfusion is called a hematocrit. The hematocrit measures the ratio of red cells to plasma. Samples are taken from a large vein. The hematocrit, however, only measures the concentration of red cells, and it is not known to measure the total amount of blood in an individual.

This hematocrit test is further complicated by the fact that if a patient has a low blood volume, called hypovolemia, which is a decrease in the volume of circulating blood or generally a low blood volume, the measured hematocrit will be artificially elevated. This is a common situation seen in patients who have lost blood from either surgery or trauma. A change in hematocrit occurs by transference of water into the plasma in the blood stream, thereby lowering the concentration of the remaining red cells within the blood. However, this process can take hours, days, or may occur incompletely. Under these circumstances, physicians may inappropriately withhold necessary blood transfusions because of the mistaken impression that the patient has more blood than he/she may actually have. This occurs because the hematocrit is artificially elevated.

Under certain circumstances, patients may retain extra salt and water and expand their blood volume (hypervolemia). Examples of this are hypertension (high blood pressure) or congestive heart failure in its early stages. Under these circumstances the hematocrit may be diluted by the increased plasma. The patient will have a lower hematocrit and may appear to be anemic.

Until recently, it has been very difficult to obtain a blood volume measurement, so that surrogate tests such as hematocrits have become and are considered the basic tests used for blood volume estimation.

It is also known that women normally have approximately twenty percent (20%) less red cells than a man of equal height and weight. Women are known to have a higher frequency of complications from certain types of surgery such as cardiac and orthopedic surgery. Women's higher mortality rates after coronary artery bypass graft surgery has been attributed to increased transfusions, as in the study “Allogeneic blood transfusions explain increased mortality in women after coronary artery bypass graft surgery,” by Rogers et al., published in the December 2006 American Heart Houmal. This invention takes this problem, which appears to be unique to women, into consideration.

As is well known, in response to the dangers of donor blood, some patients have stored their blood in the weeks blood prior to surgery. Sometimes patients suffer complications from blood donation. Among the problems of storage of blood by individuals prior to surgery is the fact that some individuals do not have a normal blood volume at the time they are donating. They may have an undetected low blood volume (hypovolemia), or they may be permitted to donate blood when it is known in advance that they are already anemic. Those patients who are already anemic may also have unrecognized hypovolemia. For example, the standard cutoff for normal healthy people to donate blood is a minimum hematocrit of 38%. In contrast, many hospitals will permit patients to donate autologous blood with a hematocrit as low as 33%. However, a patient who is 20% hypovolemic with a measured hematocrit of 33% has a true hematocrit of only 26%. Such a patient with a low or decreased hematocrit is at significant risk from the removal of a single pint of blood which is normally the standard amount or quantity for a single blood donation.

Another known present day complication is the fact that autologous blood donors are sometimes permitted to donate as much as two to six (2 to 6) pints of blood within a short period or a five (5) week period prior to surgery. The Food and Drug Administration in the United States (FDA), by contrast, will permit a healthy donor to donate only one pint of blood within eight (8) weeks of a previous donation. Under certain circumstances the red blood cells from two (2) pints may be removed and the plasma returned within an eight (8) week period. This policy, however, still results in healthy donors being able to donate much less blood than sick individuals and at a much lower frequency. Patients are unable, without special treatment, to replenish these large quantities of donated red cells. Some patients will enter surgery anemic and often with an additional burden of unrecognized hypovolemia.

There are various technologies available that can be used to decrease the possibility that an individual will require a transfusion with donor blood, or that an individual will undergo surgery or remain post-operative in a blood depleted state. One such technology is the use of the medication epoetin alfa, which stimulates the production of red blood cells. This is a known treatment for anemia but is only rarely used preoperatively.

It is also well known that blood may generally be stored prior to surgery.

Conventionally, blood is generally stored in two ways: either by refrigeration of donated blood, or freezing. Blood is also conventionally stored in mechanical freezers at a temperature of minus eighty degrees centigrade (−80° C.), and provided with a liquid nitrogen backup system in the event of loss of electricity.

Refrigerated blood has a maximum life span of approximately forty-two (42) days. Separated red cells can be stored for forty-two (42) days, while whole blood can only be stored for thirty-five (35) days. Refrigerated blood has the potential, under rare circumstances, of developing infection within a patient's own blood from bacteria that are initially present in very tiny quantities. The longer blood is stored (up to its ultimate expiration point) the more likely such an event can occur. The advantage of storing blood prior to surgery, five (5) weeks in advance, is partially counterbalanced by the small but potential risk of infection from the patient's own blood.

Another major disadvantage of refrigerated blood is that refrigerated blood shows significant loss of oxygen carrying capacity within fourteen days, and continued deterioration. This deterioration does not occur when blood is frozen soon after collection. Therefore, the benefit that a patient obtains by storing blood four to five weeks before surgery is partially lost and counter-balanced by the deterioration of the patient's blood during refrigerated storage.

Another method of storing a patient's blood is the utilization of freezing blood. Conventionally, frozen blood is normally stored as components; specifically, plasma is separated from the red cells and then frozen. The red cells are then, under sterile conditions, mixed with a special cryopreservative to enable the cells to withstand extremely low temperatures and then separately frozen. Frozen blood has several important advantages. These include no potential for bacterial growth as compared to refrigerated blood, and no loss of certain key enzymes within the red blood cells. Older or refrigerated blood does not carry oxygen (O₂) as well as well as frozen blood, which retains these enzymes. Up to 75% of oxygen (O₂) carrying capacity may be lost in blood refrigerated for thirty-five days. These changes are avoided by freezing blood within six (6) hours after collection. Frozen blood has the disadvantage of being technically difficult to perform and is performed in only a limited number of facilities in the US. It is significantly more expensive than refrigerated blood and does require more complex preparation prior to freezing as compared to the refrigeration of blood. The extra costs are far outweighed by the patient's benefit of available autologous blood and avoidance of surgery in a blood depleted state, as well as avoidance of a transfusion with deteriorated autologous refrigerated blood. A proven advantage of frozen blood prepared in accordance with the method of this invention, as will be explained, is that it can be stored for about ten (10) years with minimal change.

Red blood cells are conventionally frozen using a cryopreservative which is obtainable from many sources, and they are thawed which enables the red cells to be used up to 14 days after thawing.

The purpose of the autologous section of the blood optimization program is to ensure that patients will enter surgery in a non-anemic state and will have the safest possible blood available (primarily autologous) in the event that a transfusion is needed.

An advanced form of donor (non-autologous) blood (if needed), is within the purview of this invention and includes:

A. multi units from a single donor is within the purview of this invention;

B. avoidance of cross reactions between units of blood from different donors. By using a single donor wherever possible and, if necessary, cross-matching blood from multiple donors against each other. Donor blood is always cross-matched to a recipient to detect cross reactions between the donor blood and the recipient's blood which may be undetectable by routine blood typing, However, when multiple donors are used, the different donor bloods may cross react within the patient's body because different donor bloods are not cross checked with each other; and

C. Doubly tested blood donor units: In many ways similar to the art of sperm banking where many diseases such as HIV, various types of hepatitis, will not produce detectable antibodies for up to six (6) months in infected individuals (and occasionally even longer), and it is this reason the sperm banking industry utilizes the concept of initial testing followed by frozen storage of the donated specimen, and retesting of the individual six (6) months later to cover the period of undetectability; therefore, the concepts of the sperm banking industry can be applied to blood storage or optimization in accordance with the teachings of this invention.

Blood volume measurement is well known; however, its use in this invention is important. One of the most difficult decisions a physician faces is knowing whether to administer a transfusion to a patient who has suffered severe blood loss, particularly in situations of sudden blood loss, such as trauma, hemorrhage or surgery.

For preoperative optimization of blood volume, blood volume measurement provides an accurate way for physicians to determine the blood volume status of a patient who is planning to enter surgery, and decide on appropriate treatment in order to prevent the patient from entering surgery in an anemic or hypovolemic state. The most accurate method of measuring volume is radioisotopic blood volume measurement using the indicator dilution technique with radio iodinated Albumin I¹³¹. Blood volume provides physicians with two important pieces of information. It measures the amount of blood in the patient's body with an accuracy of ninety-eight percent (98%), and determines the amount of blood that an individual should have for their body size. For the first time, physicians have access to technology which enables them to obtain blood volume measurements rapidly and with a high degree of precision, and this forms a requirement for the proper optimization of blood for subsequent use.

The most common blood tests, the hemoglobin and hematocrit tests are used to estimate, rather than directly measure, blood volume. These tests measure the concentration of red blood cells in the blood.

The hematocrit is the standard test used to diagnose anemia, a condition in which the body has too few blood cells to adequately supply oxygen to all the tissues. The hematocrit measures the percentage, or ratio of red blood cells to plasma. It provides no information about the total volume of red blood cells, but only about their volume in relation to the amount of plasma, and this is a requirement for the optimization of blood.

Previous methods of radioisotopic blood volume measurement have required 4-6 hours for completion, but accurate results can be obtained in less than an hour with an FDA-approved semi-automated radioisotopic blood volume measurement system, the BVA-100, produced by Daxor Corporation, Empire State Building, NY, N.Y., and available at 42 medical centers throughout the country. The BVA-100 provides a measurement of whole blood, red cell, and plasma volume with an accuracy of 98% and also determines the amount of blood that each individual should have based on their body size. For the first time, physicians have access to a technology which enables them to obtain blood volume measurements rapidly and with a high degree of precision, and this enables optimization of blood for donation and surgery.

Some individuals may have a normal hematocrit and still be anemic. If both plasma and red blood cells are depleted, then the ratio of red blood cells to plasma will be normal, but the total volume of red blood cells will be low. This form of “hidden anemia” can be accurately diagnosed with a BVA-100 apparatus, which provides a measurement of the patient's total blood volume, plasma volume, and red blood cell volume. This BVA-100 apparatus is a preferred system for those who use it and need it.

The features of the BVA-100 make the blood optimization program as described to be feasible. For example, a patient who is a candidate for autologous blood donation can set up an appointment for donation and, at that appointment, receive a blood volume measurement. The patient can then wait 20-40 minutes to learn if it is safe to donate and then, if it is safe, go ahead with the donation. With previous methods of measuring blood volume, the 4-6-hour turnaround time for blood volume results meant that same-day blood volume measurement and blood donation would not be possible. Previously, blood volume measurement has rarely if ever been used prior to blood donation. The BVA-100 makes this a practical possibility.

Patients with hidden anemia may be severely ill because of inadequate treatment. Patients with chronic disease, cancer, or HIV frequently have hidden anemia.

It has always been a problem to assure that autologous blood as well as non-autologous or allogenic blood is safe, and with this invention there is an advance to such assurance. A feature of this invention in connection with the optimization of blood is in connection with storage of blood. It is preferred according to the invention to use frozen autologous blood whenever possible. In the event that autologous blood is not available, the use of multiple matched units of frozen blood from a single donor is preferred.

To these ends, the present invention consists in the provision of a method of optimization of blood of an individual comprising obtaining a unique blood volume measurement from the individual, administering an epoeten alfa medication to the individual for stimulating bone marrow to increase production of red blood cells in accordance with information obtained from the measurement of the blood volume of the individual, and storage of the blood obtained from the individual in a frozen state.

The invention, as noted, also takes into consideration that women normally have twenty percent (20%) less red cells than a man of equal height and weight. Women, generally, are known to have a higher frequency of complications from certain types of surgery such as cardiac and orthopedic surgery, and may also require more blood transfusions.

The inventive method as described herein provides an innovative approach that involves a number of technologies that have never previously before been combined into a single comprehensive program or system. The combination of some features and technologies while currently used separately when combined provide a tremendous advantage in connection with the use of obtained blood from different sources when and where necessary and enables a significant reduction in blood donor exposure and simultaneously significantly improves the chances that an individual will not be operated upon in a blood depleted state or remain in a blood depleted state post-operatively.

The key technology and method involved in this program is the use of the following technologies:

1. Utilization of Blood Volume Measurement, and this concept which has been available for about sixty (60) years, but only in the past three to five years has the FDA approved medical technology become available and which enables a rapid routine blood volume measurement to be obtained with a high degree of accuracy, heretofore, the most common method of obtaining blood volume measurements required six to eight (6-8) hours of technician time. A new system and method developed by the Applicant permits preliminary blood volume measurements to be obtained within eighteen (18) minutes, and final measurements to be obtained within thirty-five to forty-five (35 to 45) minutes. The method also provides for a blood volume measurement accurate to within ninety-eight percent (98%). An injection collection lcit enables a patient to have a blood volume measurement taken with as many as seven (7) collected samples and a separate injection of a tracer with only a single venopuncture as compared to heretofore conventional eight to ten (8 to 10) venopunctures that might be required for an equal level of accuracy.

2. Use of epoeten alfa which is a known medication that stimulates the bone marrow to increase the production of red blood cells. The use of this medication can be beneficial to individuals who are red cell volume depleted. Overstimulation of the bone marrow may result in an increase of the red cell volume to levels that may cause a significant increase in the viscosity of the blood and may cause serious complications such as a stroke or heart attack. Use of this medication is commonly or conventionally administered on an empiric basis without knowing the patient's actual blood volume and is based primarily on measurements of the hematocrit. Blood volume measurement can help prevent inappropriate therapy.

One of the problems with the use of erythropoietin has been an increased risk of tlirombotic episodes (including heart attack or stroke) following therapy (reference below). This may occur because the patient's red blood cell volume, rather than rising to a normal range in response to treatment, becomes too high. With blood volume measurement, physicians can use the patient's measured red cell volume to

a) determine if any treatment is needed at all, and

b) decide on an appropriate initial dose. For example, a patient with a mild anemia would receive a smaller initial dose, and a patient with a more severe anemia might be given a higher initial dose. The physician can then evaluate the patient's response to treatment to titrate to an optimal dose for that patient. At the present time, the usual practice is to give a standard (one size fits all) dose.

Studies which may be of interest are set forth in Bohlius J, Wilson J, Seidenfeld J, et al. Recombinant Human Erythropoietins and Cancer Patients: Updated Meta-Analysis of 57 Studies Including 9353 Patients. J Natl Cancer Inst. 2006 May 17;98(10):708-14.

The storage of blood is an important part of the procedure, and blood is conventionally stored by individuals prior to surgery, and donor blood can also be stored, with the period of storage determined by the procedure used for the storage of blood. In this invention, the use of frozen blood storage is preferred, although refrigerated blood storage may be utilized if it is determined to be preferable to frozen blood storage in a given situation.

A feature of the invention is the uniform use of filters to remove white cells and decrease the chance of transfusion reaction and transfusion of cytomegalovirus (CMV) and Epstein Barr virus in the event that door blood is required. White cells may contain infectious viruses. Filters virtually eliminate this risk. Currently, filters to remove white blood cells are not always used. In the blood optimization program, their consistent use for donor blood is part of the goal of optimizing the safety of blood products. It should be noted that white blood cells are not filtered out of autologous blood, as one's own white blood cells are beneficial.

By combining these various components, a blood optimization program or method and system is provided which, it is expected, will significantly reduce the possibility that a person will require a transfusion, or that a person will be operated upon while in a blood depleted state or remain in a blood depleted state after surgery.

Other features of the invention include, for non-autologous blood, ensuring that CMV-negative recipients receive CMV-negative door blood and the use of multi-unit, single-donor platelets.

While other blood volume measurement systems exist, the teachings of this invention provide an advanced system to assure little harm to a blood receiver or donee. Blood volume measurement is an integral part of the therapy of utilizing blood transfusions as well as having patients donate blood for themselves prior to surgery. When used in conjunction with are accepted treatments for red cell depletion such as epoetin alfa, also known as Epogen and Procrit and manufactured by Amgen and Johnson & Johnson respectively, patients can be treated to decrease the likelihood of undergoing surgery in a severely anemic state. Blood volume measurement is a critical key and forming part of the procedure according to the invention and is used for treatment decisions to detect low blood volume and help physicians plan optimal treatment.

Avoidance of blood transfusions for hypovolemia with particular emphasis on women as noted heretofore, women have been shown to require more transfusions and have a higher incidence of complications from cardiac bypass surgery. By utilizing the procedure according to the invention, it is possible to screen both men and women pre-operatively for treatment with medication to build up their blood volume to normal prior to surgery. Patients who have a normal blood volume at the onset of surgery are less likely to require transfusions and less likely to have inadequate blood flow to their brain.

A blood volume measurement, as noted, is important because it can provide specific information about the extent of red cell depletion.

The system and method according to the invention provides for autologous blood storage prior to elective surgery combined with blood volume measurement for optimal therapy.

An advantage of this invention in connection with blood volume measurement is that blood volume measurement can help to prevent inappropriate therapy, particularly in connection with the measurement of hematocrit to provide a more accurate dosage of the quantity of the epoeten alfa to be administered.

A major advantage of the present invention is the availability of frozen autologous blood and, in the event that insufficient autologous blood is available, the potential for the use of multiple matched units of doubly tested frozen blood from a single donor.

Steps for the Blood Optimization Program which includes Presurgical Autologous Blood Donation and Blood Volume Optimization:

A. Perform blood volume measurement and establish patient's blood volume status, and check for anemia:
i. If the patient is anemic (red cell volume more than 10% depleted), treat the patient with epoetin alfa and other appropriate treatments to increase the red cell volume. After a normal red cell volume has been achieved, the patient will donate a unit of blood to be prepared and stored in a frozen state; and
ii. If the patient is not anemic, the patient will donate a unit of blood to be frozen and stored.
   B. After blood donation, all patients are treated with Epogen and, if appropriate, iron to speed up replenishment of any lost blood.
   C. Depending on the surgical procedure that is going to be performed, the amount of time before surgery, and the projected anticipated amount of expected blood loss, the patient may donate 2-5 units of blood. After each donation, blood replenishment should be supplemented with epoetin alfa. If multiple units are taken, a repeat blood volume measurement may be indicated.
   D. The patient should enter surgery only after having had time to replenish blood from the patient's most recent donation. The patient's stored blood should also be available in case it is needed for transfusion.

Blood Optimization for Allogeneic Blood Donation (Donor Blood), should include the following:

A. Follow standard safety procedures for blood donation by a healthy individual.
B. A donor donates one unit of blood, which is then prepared and stored in a frozen state. Preparation includes screening of the blood for infectious diseases. Additional tests are performed, such as for cytomegalovirus (CMV), West Nile Virus, white blood count, and liver function, which are currently not required for standard blood donation, would also be performed.
C. The donor may donate additional units of blood after approximately two months. Each donated unit is tested.
D. After donating blood, the donor must wait at least six months. After a six month period of time, the donor gives a sample of blood to be tested for infectious diseases.
E. Only after that second testing is the blood cleared to be used as donor blood. This only applies to blood that was donated six or more months before the second testing.
F. If a single donor donates multiple units of blood, that blood is labeled and stored so that transfusion of multiple units can be performed from a single donor.

The goal of the allogeneic section of the blood optimization program is to ensure that patients who are unable to use autologous blood or who need more blood than they have stored can receive the safest possible donor blood for transfusion. This section is a supplement to the autologous section of the program. While it could potentially stand alone as a way to improve donor blood safety, it is most effective when incorporated into a larger blood optimization program.

The procedure and program including the method and system may be applied to mammals, although the details of treatment may differ depending on the mammal. Blood volume measurement has been performed on dogs at Columbia-Presbyterian as part of heart failure studies.

### DETAILED DESCRIPTION OF THE INVENTION

The present invention requires the following steps to carry out the optimization of blood for use in individuals requiring blood.

The procedure which includes the system and method to be carried out for the optimization of blood for future use is to be practiced in accordance with the teachings of this invention.

Utilization of Blood Volume Measurement with current medical technology which enables rapid routine blood volume measurement to be obtained with a high degree of accuracy. The method and system developed by the Applicant permits preliminary blood volume measurements to be taken within eighteen (18) minutes after injection of the tracer, and final measurements to be made within thirty-five (35) to forty-five (45) minutes. The system and method also provides blood a volume measurement accurate to within ninety-eight (98) percent. The injection procedure enables a patient to have a blood volume measurement with as many as seven (7) collected samples and a separate injection of the tracer with only a single venopuncture as compared to eight (8) to ten (10) venopunctures that might be required for an equal level of accuracy, if the other steps of this procedure are followed.

Use of eoetin alfa which is referred to heretofore as a known medication is also preferred in the procedure according to the invention. As known, epoeten alfa is a medication which stimulates the bone marrow to increase the production of red blood cells. The use of this medication as noted heretofore is beneficial to individuals whose red cell volume is depleted. To avoid a stroke or heart attack, this medication should not be administered on an empiric basis or based primarily on measurements of the hematocrit without knowing the patient's actual blood volume. As noted heretofore, blood volume measurement can help prevent inappropriate therapy.

The next step is the storage of blood prior to surgery. This is an important element in this procedure. Blood storage is accomplished primarily in two (2) ways, either by refrigeration of donated blood, or freezing. Refrigerated blood can develop infection within a patient's own blood from bacteria that are initially present in very tiny quantities. Autologous blood should be frozen whenever possible.

An additional feature to overcome the disadvantage of refrigerated blood is to freeze the blood since deterioration does not occur when blood is frozen soon after collection. It is preferred to freeze the blood within six (6) hours after collection.

It is also desirable to store frozen blood based on its individual components. Therefore, the blood is to be stored as components, and the plasma is to be separated from the red cells and then frozen. Fresh frozen plasma can be stored for one (1) year. Frozen plasma, which has less effective clotting, can be stored for ten (10) years.

After separation, the red cells are then mixed with a special cryopreservative, or antifreeze, under sterile conditions and then separately frozen. Red blood cells can be stored for ten (10) years with minimal change.

With the system and method according to the invention, it is possible to use filters to uniformly remove white cells and decrease chance of transfusion reaction and transfusion of CMV and Epstein Barr virus. White cells may contain infectious viruses. Filters eliminate this risk. Some blood banking services use filters and some do not. In the Blood Optimization procedure, filters would always be used for non-autologous blood to remove white cells which not only cause infections, but may cause low level transfusion reactions because they are not cross matched.

These components when combined make it possible to carry out the optimization of blood and provide for a procedure which significantly reduces the chance that a person will require a transfusion, or that they will be operated upon or remain in a blood depleted state after surgery, or for that matter require non-autologous blood. The blood optimization program procedure or process incorporates steps so that in the event donor blood is required, the level of donor exposure is significantly reduced.

As an initial step in the blood optimization process it is desirable to determine the approximate amounts of blood to be utilized in an impending surgical procedure. In fact, such initial determination may be very helpful in connection with the process.

Some medical procedures, such as cardiac bypass surgery, in addition to resulting in blood loss, require dilution of the patient's own blood to provide the heart bypass instrument with blood for pumping purposes. It is not uncommon to dilute the patient's blood to levels that are 50% below normal. This type of dilution is used even in patients who have major coronary and cerebral artery disease.

A recent report in the New England Journal of Medicine reported that 50 to 80% of the patients undergoing cardiopulmonary bypass surgery experience some degree of permanent memory loss. There is a significant likelihood that performing surgery on these patients with their blood markedly diluted plays a role in this memory loss. Use of the procedure according to the invention may prevent or reduce this type of exposure to extreme anemia.

Once a patient's approximate blood requirement has been determined, the next operational step is to obtain a blood volume measurement on the patient. If the patient has a normal blood volume, the patient will donate blood for autologous storage. Depending on the anticipated amount of blood required, this donated blood can either be stored in a refrigerated or frozen state. If a large quantity of blood is required, then the blood should be frozen so that repeat donations can be obtained at a frequency which will enable the patient adequate time to restore their blood volume and red cell volume to normal at the time of surgery.

Use of bone marrow/blood cell stimulant (Epoietin Alfa)—This is a key concept within the blood optimization process and program. Every patient who has donated blood should receive a dosage of Epogen. The dosage of Epogen (Epoieten Alfa) will depend on the rate at which a patient's donations are planned, and also in relation to the patient's initial blood volume. Other factors to be considered are the patient's age. Some patients may require larger and more frequent injections to permit restoration of blood volume to normal prior to surgery. For patients requiring large quantities of blood, (as much as 4 to 6 units for example, in hip surgery), a repeat blood volume may be required to verify that the patient is receiving a dose of Epoetin-alfa which permits restoration of blood volume.

Knowledge of the patient's initial blood volume is essential for achieving blood optimization. This can be summarized as follows:

Goals accomplished with the present invention:

A. Measurement of a patient's blood volume to determine what will be needed to replenish blood lost through autologous donation and to achieve normal volumes of blood—(red cells and plasma) at the time of surgery
B. preparation and priming of bone marrow with epoetin alfa to heighten rate of red cell production at time of surgery; and
C. storage of blood in a frozen state to avoid deterioration of red cells associated with refrigeration storage and to permit replacement of blood to normal levels during and after surgery.

Additional Steps if Donor Blood is Needed

A. Use of multi unit single donor blood;

B. use of doubly tested donor blood which is stored for a period of at least six (6) months between donation and testing to eliminate window period of non-detectability;

C. matching CMV-negative recipients to CMV-negative donors;

D. single donor platelets; and

E. Uniform use of filters for donated blood.

While there has been described what is considered to be the preferred system, method, process and procedure for carrying out the optimization of blood, it will be obvious to those skilled in the art that various changes and modification may be made without departing from the scope of the invention.

1. A method of optimization of blood of an individual comprising:
obtaining a unique blood volume measurement from the individual;
administering an epoeten alfa medication to the individual for stimulating bone marrow to increase production of red blood cells in accordance with information obtained from the measurement of the blood volume of the individual; and
storage of blood obtained from the individual in a frozen state.

2. The method as claimed in claim 1, wherein the blood is stored and refrigerated for a period up to forty-two (42) days.

3. The method as claimed in claim 1, including determining the approximate amount or quantity of blood to be utilized in a surgical procedure.

4. A method for the optimization of blood of an individual for self-use or for donor use as claimed in claim 1, including:
matching of CMV-negative recipients to CMV-negative donors.

5. The method as claimed in claim 4, ensuring that in addition to CMV-negative recipients receive CMV-negative donor blood, for non-autologous blood the use of multi-unit, single donor platelets.

6. The method as claimed in claim 1, including uniform use of filters for blood of the individual who acts as a donor to provide donee blood.

7. The method as claimed in claim 1, including use of single donor platelets.

8. The method as claimed in claim 1, including use of a multi-unit single donor blood.

9. The method as claimed in claim 1, including use of doubly tested donor blood which is stored for a period of at least six (6) months between donation ad testing to eliminate a window period of non-detectability.

10. The method as claimed in claim 1, wherein preliminary blood volume measurements are obtainable within eighteen minutes and final measurements are obtainable within thirty-five to forty-five minutes after commencement of administration of the epoetin alfa medication.

11. The method as claimed in claim 1, wherein the measurement of blood volume of an individual is effective to provide an indication of the amount of blood in the body of the individual with an accuracy of approximately ninety-eight percent (98%), and determine the amount of blood that an individual should have for their body size.

12. The method as claimed in claim 10, wherein the epoetin alfa, should only be administered when the patient's actual blood volume is known.

13. A system for customizing blood for future use comprising a combination of the following procedures:
obtaining a blood volume measurement of the blood volume from a potential blood donor;
administering an epoetin alfa medication to the potential blood donor for stimulating bone marrow of the potential blood donor to increase red blood cells of the potential blood donor in accordance with information obtained from the measurement of the blood volume of the potential blood donor;
obtaining blood from the potential blood donor if the blood donor is qualified in accordance with acceptable medical procedures to donate blood; and
storage of the blood obtained in a frozen state.

14. The system as claimed in claim 13, including providing and using filters to remove white cells from donated blood.

15. The system as claimed in claim 13, including performing a match to match CMV-negative recipients to CMV-negative donors.

16. The system as claimed in claim 13, wherein use is made of doubly-tested donor blood which is stored for a period of at least six (6) months between donation and testing to eliminate a window period of non-detectability.

17. The system as claimed in claim 13, including use of a collection kit to enable patients' blood to be taken with a single venopuncture.

18. The system as claimed in claim 13, including carrying out tests for cytomegalovirus (CMV), West Nile virus, white blood count and liver function.

19. The system as claimed in claim 12, wherein the measurement of blood volume of an individual is effective to provide an indication of the amount of blood in the body of the individual with an accuracy of approximately ninety-eight percent (98%), and determine the amount of blood that an individual should have for their body size.

20. The system as claimed in claim 13, wherein the epoetin alfa, should only be administered when the patient's actual blood volume is known.
